(19) United States
(12) Patent Application Publication
(10) Pub. No.: US 2010/0131002 A1
(43) Pub. Date: May 27, 2010

(54) Stent with a net layer to embolize and aneurysm

(76) Inventors: Robert A. Connor, Minneapolis, MN (US); Muhammad Tariq Janjua, Inver Grove Heights, MN (US)

Correspondence Address: Robert A. Conner, 100 Third Ave. S., Unit #304, Minneapolis, MN, 55401, US

(21) Appl. No.: 12/592,116

(22) Filed: Nov. 18, 2009

Related U.S. Application Data
(60) Provisional application No. 61/200,093, filed on Nov. 24, 2008.

(51) Int. Cl. A61F 2/01

(52) U.S. Cl. 606/200

(57) ABSTRACT

This invention is a stent that is inserted into the parent vessel of an aneurysm in order to reduce blood flow to the aneurysm and promote embolization of the aneurysm. The stent wall includes an inner structure that can be expanded from a compressed state to a resilient expanded state and an outer flexible layer that covers all or part of the inner structure. Embolic members are placed and retained in the gap between the inner structure and the outer layer in the area of the aneurysm neck in order to reduce blood flow to the aneurysm.

### CROSS-REFERENCE TO RELATED APPLICATIONS:

This patent application claims the priority benefits of U.S. Provisional Patent Application Ser. No. 61/200,093 entitled “Stent with a net layer to embolize an aneurysm” filed on Nov. 24, 2008 by Robert A. Connor.

### FEDERALLY SPONSORED RESEARCH: Not Applicable

### SEQUENCE LISTING OR PROGRAM: Not Applicable

### BACKGROUND—FIELD OF INVENTION

This invention relates to devices to treat aneurysms.

### BACKGROUND AND REVIEW OF RELATED ART

An aneurysm is an abnormal bulging or ballooning of a blood vessel. Rupture of brain aneurysms can cause stroke, death, or disability. Around one-third of people who have a brain aneurysm that ruptures will die within 30 days of the rupture. Of the survivors, around half of them suffer some permanent loss of brain function. Many aneurysms are not identified until they rupture. However, identification of intact aneurysms is increasing due to increased outpatient imaging. Ruptured aneurysms must be treated to stop the bleeding or to prevent re-bleeding. Intact aneurysms may or may not be treated to prevent rupture, depending on their characteristics. Wide neck aneurysms are less prone to rupture, but are harder to treat. In the U.S., it has been estimated that over 10 million people have brain aneurysms and 30,000 people each year have a brain aneurysm that ruptures.

Several approaches can be used to treat brain aneurysms. These different approaches can be divided into three categories: (1) approaches involving treatment outside the vessel; (2) approaches involving treatment inside the aneurysm; and (3) approaches involving treatment in the parent vessel. Some of these approaches can be used together. Each of these approaches has some disadvantages, as discussed below.

1. Treatment Outside the Vessel

Clipping: Clipping is the application of a small clip to the aneurysm neck from outside the vessel to seal off the aneurysm. For most brain aneurysms, this involves invasive surgery including removing a section of the skull. Clipping began in the 1930's and is well-established. Clipping is more common in the U.S. than in Europe. Around half of all aneurysms are treated by clipping. There are many aneurysm clips in the prior art. However, due to its invasive nature, clipping is decreasing. Potential disadvantages of clipping can include: significant health risks associated with major surgery of this type; and long recovery times, even when the surgery itself goes well.

2. Treatment Inside the Aneurysm

Metal Coils: Metal coiling is the endovascular insertion of metal coils into the aneurysm to reduce blood flow and promote embolization in the aneurysm. Historically, metal coils have been platinum. Coils are more common in Europe than in the U.S. There are many examples of metal coils. Potential disadvantages of metal coils can include: low percentage of aneurysm volume filled (low occlusion is associated with a higher risk of rupture); compaction of coils over time; risk of recanalization; potential prolapse of coils into the parent vessel; difficulty later clipping aneurysms filled with metal coils, if needed; pressure from the coils on surrounding brain tissue; inability of coils to treat all aneurysms; and expense of metal coils (especially platinum coils).

Combination Metal/Textile/Foam/Gel Coils: Coils with a combination of metal and other materials can be used to try to achieve greater occlusion volume than metal coils alone. These other materials include textile, foam, and gel elements. Textile strands can be woven into the coils to add bulk. Coils can be covered with soft foam. Gel elements can be strung together into elongated structures. Examples of related art that appear to use this approach includes the following: U.S. Pat. Nos. 5,382,259 (Phelps et al.), 5,522,822 (Phelps et al.), 5,690,666 (Berenstein et al.), 5,718,711 (Berenstein et al.), 5,749,894 (Engelson), 5,976,162 (Doan et al.), 6,024,754 (Engelson), 6,299,619 (Greene, Jr. et al.), 6,602,261 (Greene, Jr. et al.), 6,723,108 (Jones et al.), 6,979,344 (Jones et al.), 7,070,609 (West), and 7,491,214 (Greene, Jr. et al.), and U.S. Patent Applications 20040158282 (Jones, Donald et al.), 20050267510 (Razack, Nasser), and 20060058834 (Do, Hiep et al.). Potential disadvantages of combination coils can include: remaining gaps between loops; compaction of coils over time; risk of recanalization; potential prolapse of coils into the parent vessel; difficulty clipping aneurysms filled with coils with metal components later if needed; pressure from the coils on surrounding brain tissue; inability of coils to treat all aneurysms; and expense of metal coils.

Inflatable Balloons: Approximately two decades ago, there were numerous efforts to treat aneurysms by permanently filling them with inflatable balloons. These efforts were largely abandoned due to the risks of balloon deflation, prolapse into the parent vessel, aneurysm rupture, and recanalization. There are, however, examples of relatively recent art that appear to use inflatable balloons to treat aneurysms: U.S. Pat. Nos. 6,569,190 (Whalen et al.) and 7,083,643 (Whalen et al.), and U.S. Patent Applications 20030135264 (Whalen et al.), 20030187473 (Berenstein, Alejandro et al.), 20060292206 (Kim, Steven et al.), 20070050008 (Kim, Steven et al.), and 20070055355 (Kim, Steven et al.). Potential disadvantages of using inflatable balloons to permanently fill aneurysms can include: balloon deflation; prolapse of the balloon into the parent vessel; aneurysm rupture due to balloon pressure; and recanalization.

Manually-Activated Mesh Occluders: Another approach to treating aneurysms involves inserting into the aneurysm a mesh structure, generally metal, that can be expanded or contracted by human-controlled mechanical motion so as to block the aneurysm neck and/or to fill the main volume of the aneurysm. For example, a wire structure can be inserted through the aneurysm neck in a narrow configuration and then transformed into an “hour-glass” shape that collapses to block the aneurysm neck when activated by a human controller. Examples of related art that appear to use this approach include the following: U.S. Pat. Nos. 5,928,260 (Chin et al.), 6,344,048 (Chin et al.), 6,375,668 (Gifford et al.), 6,454,780 (Wallace), 6,746,468 (Sepetka et al.), 6,780,196 (Chin et al.), and 7,229,461 (Chin et al.), and U.S. Patent Applications 20020042628 (Chin, Yem et al.), 20020169473 (Sepetka, Ivan et al.), 20030083676 (Wallace, Michael), 20030181927 (Wallace, Michael), 20040181253 (Sepetka, Ivan et al.), 20050021077 (Chin et al.), 20060155323 (Porter, Stephen et al.), 20070088387 (Eskridge, Joseph et al.), 20070106311 (Wallace, Michael et al.), and 20080147100 (Wallace, Michael). Potential disadvantages of such manually-activated metal occluders include: difficulty engaging the necks of wide-neck aneurysms; difficulty filling irregularly-shaped aneurysms with standard-shaped mesh structures; risk of rupture when pinching the aneurysm neck or pushing on the aneurysm walls; and protrusion of the proximal portion of “hour-glass” designs into the parent vessel.

Self-Expanding Standard-Shape Occluders: Another approach to treating aneurysms uses standard-shaped structures that self-expand when released into the aneurysm. For example, the structure may be a mesh of “shape memory” metal that automatically expands to a standard shape when released from the confines of the catheter walls. As another example, the structure may be a gel that expands to a standard shape when exposed to moisture. Examples of related art that appear to use this approach include the following: U.S. Pat. Nos. 5,766,219 (Horton), 5,916,235 (Guglielmi), 5,941,249 (Maynard), 6,409,749 (Maynard), 6,506,204 (Mazzocchi), 6,605,111 (Bose et al.), 6,613,074 (Mitelberg et al.), 6,802,851 (Jones et al.), 6,811,560 (Jones et al.), 6,855,153 (Saadat), 7,083,632 (Avellanet et al.), 7,306,622 (Jones et al.), and 7,491,214 (Greene, Jr. et al.), and U.S. Patent Applications 20030093097 (Avellanet, Ernesto et al.), 20030195553 (Wallace, Michael et al.), 20050033349 (Jones, Donald et al.), 20060052816 (Bates, Brian et al.), and 20060235464 (Avellanet, Ernesto et al.) and WIPO Patents WO/2006/084077 (Porter, Stephen et al.) and WO/1996/018343 (McGurk et. al.). Potential disadvantages of such self-expanding standard-shape structures can include: risk of prolapse into the parent vessel, especially for wide-neck aneurysms; difficulty occluding irregularly-shaped aneurysms with standard shape structures and associated risk of recanalization; and difficulty generating the proper amount of force (not too much or too little) when engaging the aneurysm walls with a standard-shaped self-expanding structure.

Self-Expanding Custom-Modeled Occluders: A variation on self-expanding standard-shape occluders (discussed above) are self-expanding occluders that are custom modeled before insertion so as to fit the shape of a particular aneurysm. As an example sequence—the aneurysm can be imaged, the image is used to custom model the occluding structure, the occluding structure is compressed into a catheter, the occluding structure is inserted into the aneurysm, and the occluding structure then self-expands to fill the aneurysm. The occluding structure may be made from a gel that expands upon contact with moisture. Examples of related art that appear to use this approach include the following: U.S. Pat. Nos. 5,766,219 (Horton), 6,165,193 (Greene, Jr. et al.), 6,500,190 (Greene, Jr. et al.), 7,029,487 (Greene, Jr. et al.), and 7,201,762 (Greene, Jr. et al.), and U.S. Patent Application 20060276831 (Porter, Stephen et al.). Potential disadvantages of self-expanding custom-modeled occluders can include: the complexity and expense of imaging and modeling irregularly-shaped aneurysms; difficulty compressing larger-size structures into a catheter; difficulty inserting the occluding structure in precisely the correct position; and difficulty getting a gelatinous surface to anchor solidly to aneurysm walls.

Congealing Liquid or Gel: Another approach to treating aneurysms involves filling an aneurysm with a liquid or gel that congeals rapidly. Examples of related art that appear to use this approach include the following: U.S. Pat. Nos. 6,569,190 (Whalen et al.), 6,626,928 (Raymond et al.), 6,958,061 (Truckai et al.), and 7,083,643 (Whalen et al.), and U.S. Patent Application 20030135264 (Whalen et al.). Potential disadvantages of a congealing liquid or gel can include: leakage of the congealing substance into the parent vessel, potentially causing a stroke; difficulty filling the entire aneurysm if the substance begins to congeal before the aneurysm is full; and seepage of toxic substances into the blood stream.

Biological or Pharmaceutical Agents: Biological and/or pharmaceutical agents can enhance the performance of a variety of mechanical treatment methods for aneurysms. For example, they can speed up the natural embolization process to occlude the aneurysm. Examples of related art that appear to use this approach include the following: U.S. Patent Applications 20060206139 (Tekulve, Kurt J.), 20070168011 (LaDuca, Robert et al.), and 20080033341 (Grad, Ygael). Currently, biological and/or pharmaceutical approaches are not sufficient as stand alone treatment approaches for many cases. Accordingly, they share most of the potential disadvantages of the baseline approach to which the biological or pharmaceutical agents are added.

Embolic-Emitting Expanding Members: Another approach involves an expanding member within the aneurysm that emits embolic elements into the aneurysm. Examples of such expanding members include bags, meshes, and nets. Examples of embolic elements include coils and congealing liquids. This can be viewed as another way to block the aneurysm neck while delivering embolics into the volume of the aneurysm. For example, the distal portion of an expanding bag may leak embolic elements into the aneurysm, but the proximal portion of the expanding member does not leak embolics into the parent vessel. Examples of related art that appear to use this approach include the following: U.S. Pat. No. 6,547,804 (Porter et al.) and U.S. Patent Applications 20040098027 (Teoh, Clifford et al.), 20060079923 (Chhabra, Manik et al.), and 20080033480 (Hardert, Michael). Potential disadvantages are as follows. Since the expanding member “leaks,” it may have insufficient expansion force to adequately anchor against the aneurysm walls or to seal off the aneurysm neck. As a result of poor anchoring, the bag may prolapse into the parent vessel. Also, as a result of poor sealing of the aneurysm neck, embolics may leak into the parent vessel.

Shape Memory Structures inside Expanding Members: A variation on the shape memory approach above involves the addition of an expanding member around the shape memory structure. Examples of related art that appear to use this approach include the following: U.S. Pat. Nos. 5,861,003 (Latson et al.), 6,346,117 (Greenhalgh), 6,350,270 (Roue), 6,391,037 (Greenhalgh), and 6,855,153 (Saadat). The potential disadvantages of this approach are similar to those for uncovered shape memory occluders: risk of prolapse into the parent vessel, especially for wide-neck aneurysms; difficulty occluding irregularly-shaped aneurysms with standard shape structures and associated risk of recanalization; and difficulty generating the proper amount of force (not too much or too little) when engaging the aneurysm walls with a standard-shaped self-expanding structure.

Accumulating Coils inside Expanding Members: A variation on the standard coiling approach above involves the addition of an expanding member around the accumulating coils. Examples of related art that appear to use this approach include the following: U.S. Pat. Nos. 5,334,210 (Gianturco), 6,585,748 (Jeffree), and 7,153,323 (Teoh et al.), and U.S. Patent Applications 20060116709 (Sepetka, Ivan et al.), 20060116712 (Sepetka, Ivan et al.), and 20060116713 (Sepetka, Ivan et al.). Potential disadvantages of this approach are similar to those for coils alone, including: compaction of coils over time; risk of recanalization due to “bumpy” coil-filled expanding member; difficulty clipping aneurysms filled with metal coils later if needed; pressure from the coils on surrounding brain tissue; inability to treat all aneurysms; and expense of metal coils (especially platinum coils).

3. Treatment in the Parent Vessel

Standard (High-Porosity) Stent: A stent is a structure that is inserted into a vessel in a collapsed form and then expanded into contact with the vessel walls. Standard stents are generally highly porous, metal, and cylindrical. A high-porosity stent allows blood to flow through the stent walls if there are any branching or secondary vessels in the vessel walls. Blood flow through a stent wall into a branching or secondary vessel is desirable, but blood flow through a stent wall into an aneurysm is not. Accordingly, a high-porosity stent in the parent vessel is not a good stand-alone aneurysm treatment. A high-porosity stent in the parent vessel can, however, help to keep coils or other embolic members from escaping out of the aneurysm into the parent vessel.

Examples of related art that appear to use this approach include the following: U.S. Pat. Nos. 6,096,034 (Kupiecki et al., 2000), 6,344,041 (Kupiecki et al., 2002), 6,168,592 (Kupiecki et al., 2001), and 7,211,109 (Thompson, 2007). Potential disadvantages of this approach can include many of the problems associated with use of the embolic members alone. For example, using a high-porosity stent in the parent vessel in combination with coils in the aneurysm still leaves the following disadvantages of using coils alone: low percentage of aneurysm volume filled (and low occlusion is associated with a higher risk of rupture); compaction of coils over time; significant risk of recanalization; difficulty clipping aneurysms filled with metal coils later if needed; pressure from the coils on surrounding brain tissue; inability of coils to treat all aneurysms; and expense of metal coils (especially platinum coils).

Uniformly Low-Porosity Stent: Another approach involves inserting a uniformly low-porosity stent into the parent vessel. The low-porosity stent blocks the flow of blood through the stent walls into the aneurysm, causing beneficial embolization of the aneurysm. For example, the stent may have one or more layers that are impermeable to the flow of liquid. Unlike a standard (high-porosity) stent, this approach can be used as a stand-alone aneurysm treatment. Examples of related art that appear to use this approach include the following: U.S. Pat. Nos. 5,645,559 (Hachtman et al., 1997), 5,723,004 (Dereume et al., 1998), 5,948,018 (Dereume et al., 1999), 6,165,212 (Dereume et al., 2000), 6,063,111 (Hieshima et al., 2000), 6,270,523 (Herweck et al., 2001), 6,331,191 (Chobotov, 2001), 6,342,068 (Thompson, 2002), 6,428,558 (Jones et al., 2002), 6,656,214 (Fogarty et al., 2003), 6,673,103 (Golds et al., 2004), 6,790,225 (Shannon et al., 2004), and 6,786,920 (Shannon et al., 2004), and U.S. Patent Application 20080319521 (Norris et al., 2008). Potential disadvantages of this approach can include: undesirably blocking blood flow to branching or secondary vessels that are close to the aneurysm and are covered by the stent wall; difficulty achieving a snug fit across the neck of the aneurysm if the parent vessel is curved, twisted, or forked; and poor attachment of the stent with the parent vessel wall due to the impermeable nature of the stent wall.

Uniformly Intermediate-Porosity Metal Stent: This approach pursues creation of a stent with a uniform intermediate porosity that provides a compromise between the benefits of a high-porosity stent in the parent vessel (good blood flow to nearby branching or secondary vessels) and the benefits of a low-porosity stents in the parent vessel (blocking blood flow to the aneurysm). Examples of related art that appear to use this approach include the following: U.S. Pat. Nos. 6,770,087 (Layne et al., 2004), 7,052,513 (Thompson, 2006), and 7,306,624 (Yodfat et al., 2007), and U.S. Patent Applications 20070207186 (Scanlon et al.,2007), 20070219619 (Dieck et al., 2007), 20070276470 (Tenne, 2007), 20070276469 (Tenne, 2007), and 20080039933 (Yodfat et al., 2008). The main potential disadvantage of this approach is that it may perform neither function very well. It may unreasonably block flow to a branching or secondary vessels (causing a stroke) and may inadequately block blood flow to the aneurysm (leaving it vulnerable to rupture).

Pre-Formed Differential Porosity Stent: This approach involves creating a stent with different levels of porosity for different wall areas, before the stent is inserted into the parent vessel. The goal is two-fold: (1) to place wall areas with high porosity over openings to branching or secondary vessels; and (2) to place wall areas with low porosity over the neck of the aneurysm. Examples of related art that appear to use this approach include the following: U.S. Pat. Nos. 5,769,884 (Solovay,1998), 5,951,599 (McCrory, 1999), 6,309,367 (Boock, 2001), 6,309,413 (Dereume et al., 2001), 6,165,212 (Dereume et al., 2000), 5,948,018 (Dereume et al.,1999), 5,723,004 (Dereume et al., 1998), and 7,186,263 (Golds et al., 2007), and U.S. Patent Applications 20070219610 (Israel, 2007), 20070239261 (Bose, et al., 2007), and 20080004653 (Sherman et al., 2008). Potential disadvantages of this approach include: difficultly matching a specific anatomic configuration (curvature, branching, neck size, etc) with a preformed stent; difficulty of precise placement of the stent to properly align the porous and non-porous areas with branching vessels and the aneurysm, respectively; and difficulty creating low porosity areas in a compressed state that maintain this low porosity in an expanded state.

Post-Implantation Filling Between Stent Wall and Vessel Wall: This approach fills the gap between the wall of the stent and the wall of the parent vessel with an embolizing substance such as a liquid or gel that solidifies after insertion. Examples of related art that appear to use this approach include the following: U.S. Pat. No. 5,769,882 (Fogarty et al., 1998) and U.S. Patent Application 20070150041 (Evans et al., 2007). Potential disadvantages of this approach include: difficulty injecting the embolizing substance through the stent wall without having it leak back into the parent vessel; leakage of embolizing liquid or gel between the stent and the parent vessel into the blood stream, where it blocks a downstream vessel and causes a stroke; challenges containing the embolic material within curving vessels or vessels with irregular walls; and difficulty using this method to fill narrow-neck aneurysms.

Post-Implantation Surface Modification: This approach creates different degrees of porosity in different wall areas after the stent is implanted. The goal is to decrease the porosity of the stent wall in the area of the aneurysm neck, but to leave the rest of the stent wall relatively porous to allow blood flow to branching or secondary members. Also, high porosity in other areas of the stent wall aids in the attachment and integration of the stent to the parent vessel. Unlike the preceding approach, this approach does not fill the gap between the stent wall and the parent vessel wall with some type of solidifying liquid, but rather modifies the wall of the stent itself. This reduces the risk of embolic liquid or members leaking out between the stent and the parent vessel wall into the blood stream.

This approach remains relatively uncommon. The few examples in the related art appear to expose one area of the stent wall to surface-modifying chemicals or energy emissions in order to decrease porosity of the stent wall in that area alone. Examples of related art that appear to use this approach include the following: U.S. Pat. Nos. 5,951,599 (McCrory, 1999) and 7,156,871 (Jones et al., 2007). Potential disadvantages of this approach include: negative effects of surface-modifying chemicals seeping into the blood stream; negative effects of energy emissions on surrounding vessel or brain tissue; and difficulty adding enough matter to the stent wall covering the aneurysm neck by chemical or energy modification means, after stent implantation, to adequately reduce blood flow through the aneurysm neck.

To conclude this section, although there has been significant progress in developing options for treating brain aneurysms, there are still high rates of death and disability and still disadvantages to the treatment options available.

### SUMMARY AND ADVANTAGES OF THIS INVENTION

This invention is a stent system that is inserted into the parent vessel of an aneurysm in order to reduce blood flow to the aneurysm and promote embolization of the aneurysm. The stent wall includes an inner structure, such as an expandable metal mesh, that can be expanded from a compressed state to a resilient expanded state and an outer flexible layer, such as a flexible fabric net, that covers all or part of the inner structure. Embolic members are placed and retained in the gap between the inner structure and the outer layer in the area of the aneurysm neck in order to reduce blood flow to the aneurysm.

This invention has several significant advantages over the current approaches to treating aneurysms, especially aneurysms in the brain. These advantages include: relatively non-invasive (especially compared to clipping); relatively high percentage of aneurysm neck blocked (especially compared to coils); relatively rapid blockage of blood flow into the aneurysm (especially compared to coils); preserves option of future clipping if necessary (especially compared to coils); low risk of puncturing aneurysm wall (especially compared to coils); low risk of recanalization (especially compared to coils and balloons); low risk of prolapse into parent vessel (especially compared to coils and balloons); low risk of deflation (compared to balloons); low risk of pinching and rupturing aneurysm neck (compared to “hour-glass” neck occluders); strengthens structure of the parent vessel (compared to intra-aneurysm approaches); selectively adjusts wall porosity in different areas after implantation (compared to conventional stents); low risk of solidifying liquid or other material escaping into blood stream and causing a stroke (especially compared to liquid embolics in the aneurysm or the gap between the stent and the parent vessel wall); no negative effects of blood-blocking chemicals leaking into the blood stream (compared to current examples of post-implantation wall modification); no negative effects of energy emissions on nearby brain tissue (compared to current examples of post-implantation wall modification); and ability to add a relatively large volume of embolic matter to the area of the stent wall covering the aneurysm neck (compared to current examples of post-implantation wall modification).

### INTRODUCTION TO THE FIGURES

FIGS. 1 through 15 show possible embodiments of this stent, but do not limit the full generalizability of the claims.

FIG. 1 shows an opaque side view of one embodiment of this stent after it has been inserted and expanded within the parent blood vessel of an aneurysm.

FIG. 2 shows an alternative view of this same embodiment, with the two layers of the stent being transparent in order to allow a clearer view of the guidewires.

FIG. 3 shows an opaque side view of this same embodiment, except that a catheter to deliver embolic members has now been slid along the guidewires to reach an opening in the inner mesh structure.

FIG. 4 shows an alternative view of this same embodiment with the two layers of the stent being transparent in order to allow a clearer view of the catheter and the embolic members.

FIG. 5 shows an opaque side view of this same embodiment, except that a plurality of embolic members have now been inserted into the gap between the inner mesh structure and the outer flexible layer in the area of the aneurysm neck.

FIG. 6 shows an alternative view of this same embodiment with the two layers of the stent being transparent in order to allow a clearer view of the catheter and the embolic members.

FIGS. 7 and 8 show this same embodiment after the detachment and withdrawal of the guidewires and catheter.

FIGS. 9 through 13 show greater detail for one example of how the guidewires and catheter function to transport embolic members into the gap between the inner mesh structure and the outer flexible layer of the stent wall.

FIG. 9 shows a close-up view of guidewires attached to the inside surface of a hexagonal opening in the inner mesh structure.

FIG. 10 shows a close-up view of the distal end of the catheter as it slides along the guidewires toward the inner mesh structure.

FIG. 11 shows a close-up view of the distal end of the catheter after it has completely slid along the guidewires to reach the inner mesh structure and be aligned with an opening in this inner mesh structure.

FIG. 12 shows a close-up view of embolic members being propelled through the catheter by a flow of sterile saline solution.

FIG. 13 shows a close-up view of a plurality of embolic members having been inserted into the gap between the inner mesh structure and the outer flexible layer, with both guidewires and catheter having been withdrawn.

FIGS. 14 and 15 show examples of this stent with a high-flexibility area of the outer flexible layer that is identified by radioopaque lines and that is positioned to cover the aneurysm neck.

### DETAILED DESCRIPTION OF THE FIGURES

FIGS. 1 through 15 show possible embodiments of this stent. However, these embodiments are not exhaustive. These figures do not limit the full generalizability of the claims.

FIG. 1 shows an opaque side view of one embodiment of this stent, after it has been inserted and expanded within the parent blood vessel of an aneurysm. FIG. 1 also shows a cross-sectional side view of the parent blood vessel 103 with aneurysm 101 including aneurysm neck 102. In this embodiment, the stent system has a resilient inner structure 104, which is a metal mesh with a hexagonal pattern, and an outer flexible layer 105 that is configured like a net around the inner structure. FIG. 1 also shows two guidewires 106 that are attached to inner structure 104. The stent is shown in FIG. 1 in an already inserted and expanded configuration. Many methods of stent insertion and expansion, such as by catheter and balloon, are well known in the art and the precise methods of insertion and expansion are not central to this invention.

In this embodiment, the wall of the stent consists of two layers. The inner layer of the stent wall is an expandable and resilient metal mesh structure 104 with a hexagonal pattern. Many other types of expandable mesh structures may also be used. In various examples, this inner mesh structure may be made from stainless steel, a nickel-titanium alloy, cobalt chromium or a cobalt-chromium alloy, titanium or a titanium alloy, tantalum or a tantalum allow, or polymeric-based resin or another polymer. In this embodiment, the outer layer of the stent is a flexible fabric net 105. In various examples, the outer flexible layer may be made from latex, nylon, polyester, teflon, silicone, HDPE, polycarbonate urethane, polyether-polyamide copolymer, polyethylene terephthalate, polyolefin, polypropylene, polytetrafluorethylene, polytetrafluoroethene, polyurethane, or polyvinyl chloride.

In this embodiment, there is a gap between the inner mesh structure and the outer flexible layer and these layers are not connected to each other. In other examples of this invention, there may be no gap between these layers until embolic members are inserted between them in the area of the aneurysm neck. In other examples, the two layers may be connected at multiple points or seams in order to form separate pouches between the layers for more precise localized containment of the embolic members between the layers. In other examples, the wall may be comprised of more than two layers.

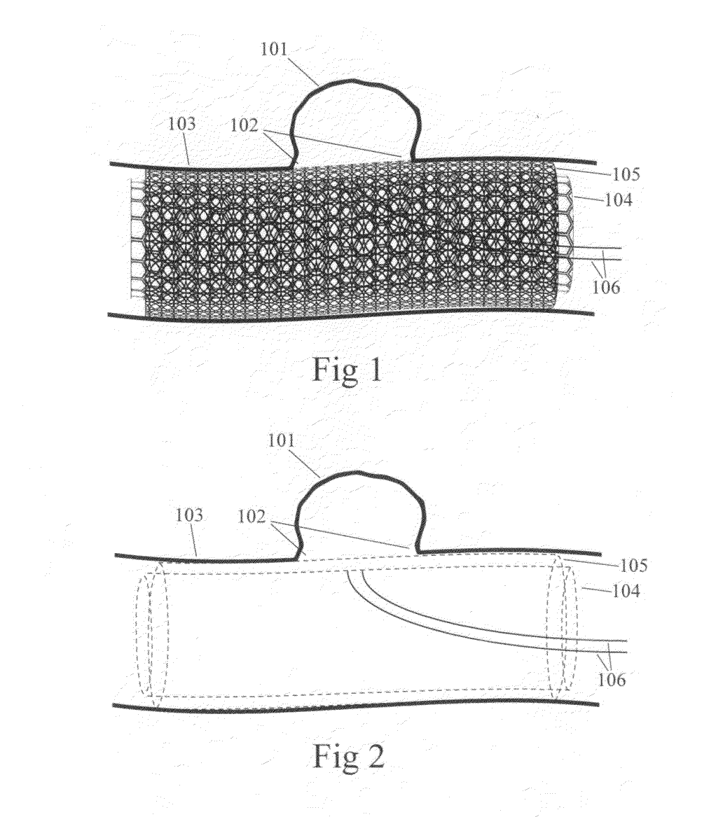

FIG. 2 shows an alternative view of the same embodiment of this stent that is shown in FIG. 1. FIG. 2 is the same as FIG. 1 except that FIG. 2 shows the two layers of the stent as transparent in order to allow a clearer view of two guidewires 106 that are attached to the inner mesh structure of the stent wall. In this embodiment, these two guidewires 106 were attached to the inner structure of the stent at a specific point before insertion of the stent and the operator has aligned this point with the aneurysm neck 102 during stent placement within the parent vessel 103. In this embodiment, these two guidewires 106 will be used to guide a catheter that delivers embolic members into the gap between the inner wall structure 104 and the outer flexible layer 105. In another example, guidewires need not be used; the catheter may be directed to the inner wall structure using real-time imaging and attached to the inner wall structure with a grasping or hooking mechanism.

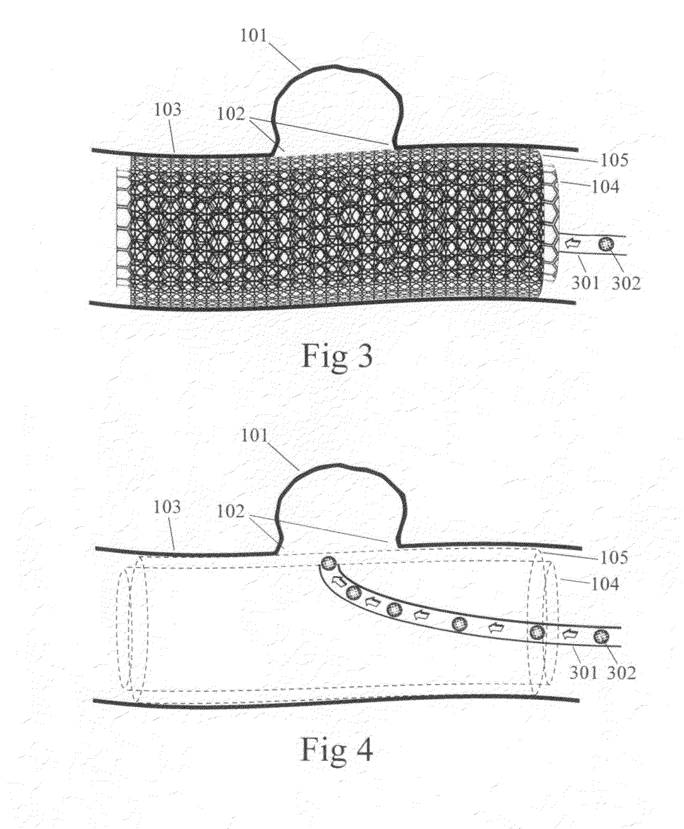

FIG. 3 shows an opaque side view of the same embodiment of this stent that is shown in FIG. 1, except that a catheter 301 to deliver embolic members (including embolic member 302) has been slid along guidewires 106 to reach an opening in the inner mesh structure 104. In this embodiment, sterile embolic members (including 302) are propelled by a flow of sterile saline solution through catheter 301 for insertion into the gap between inner mesh structure 104 and outer flexible layer 105. The saline solution propels the embolic members through the catheter and into the gap, wherein the members expand and are trapped within the gap. The saline solution escapes through the openings in the mesh. In other examples, other means may be used to transport the embolic members along the catheter, such as miniature conveyor belts or rotating helix mechanisms.

In this embodiment, the embolic members are compressible micro-sponges that expand upon ejection from the catheter. In various examples, these micro-sponges may be made from cellulose, collagen, acetate, alginic acid, carboxy methyl cellulose, chitin, collagen glycosaminoglycan, divinylbenzene, ethylene glycol, ethylene glycol dimethylmathacrylate, ethylene vinyl acetate, hyaluronic acid, hydrocarbon polymer, hydroxyethylmethacrylate, methlymethacrylate, polyacrylic acid, polyamides, polyesters, polyolefins, polysaccharides, polyurethane, polyvinyl alcohol, silicone, urethane, and vinyl stearate. In other examples, the embolic members may be gels, beads, or coils.

In this embodiment, the embolic members (such as 302) are retained with the gap between the inner mesh structure 104 and outer flexible layer 105 because they expand upon ejection from the catheter 301 and can not exit the same opening in the inner mesh structure by which they entered this gap. In another example, the embolic members need not expand, but the opening by which they enter the gap may be closed when the catheter is removed to trap them within the gap.

FIG. 4 shows an alternative view of the same embodiment of this stent that is shown in FIG. 3, except that the two layers of the stent are transparent in order to allow a clearer view of catheter 301 and embolic members (including 302).

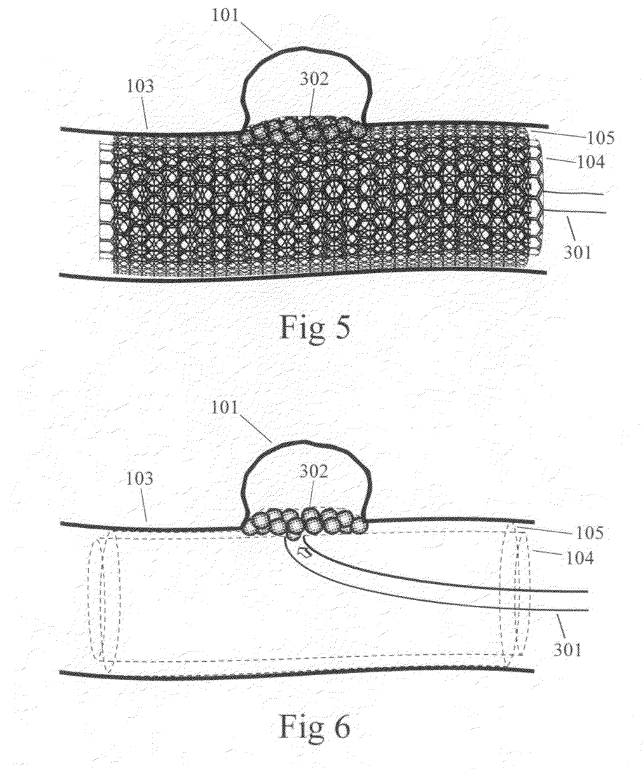

FIG. 5 shows an opaque side view of the same embodiment of this stent that is shown in FIG. 3, except that a plurality of embolic members (including 302) have now been delivered via catheter 301 and inserted into the gap between the inner mesh structure 104 and the outer flexible layer 105 in the area of the aneurysm neck. The flexibility of outer layer 105 allows it to distend into the aneurysm neck to more thoroughly block blood flow through the neck. A sufficient volume of embolic members has been inserted into this gap in the area of the aneurysm neck to occlude the flow of blood into aneurysm 101, thereby promoting embolization of the aneurysm.

FIG. 6 shows an alternative view of the same embodiment of this stent that is shown in FIG. 5, except that the two layers of the stent are transparent in order to allow a clearer view of catheter 301 and embolic members (including 302).

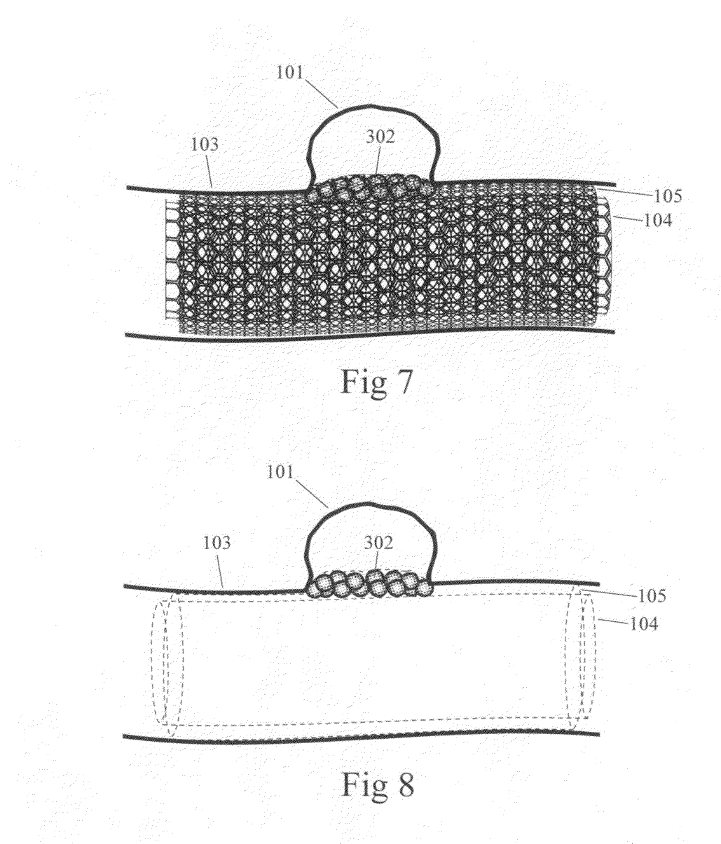

FIGS. 7 and 8 show the same embodiment, but after the detachment and withdrawal of the guidewires 106 and catheter 301. In this example, the guidewires may be detached from the inner mesh structure by application of a mild electric current and the catheter may be removed by simple mechanical withdrawal. Many other methods for detaching and removing guidewires and catheters are known in the prior art and the exact detachment and removal mechanisms are not central to this invention. Blood flow through the aneurysm neck is now largely blocked to promote embolization of the aneurysm, but other areas of the stent remain largely porous to foster integration with the walls of the parent vessel and to allow blood flow to any secondary vessels that may branch off from the parent vessel along the length of the stent.

FIGS. 9 through 13 show greater detail for one example of how the guidewires and catheter function to transport embolic members into the gap between the inner mesh structure and the outer flexible layer of the stent wall. In these figures: only small square patches of inner mesh structure 104 and outer flexible layer 105 are shown (indicated by dashed line borders); and the size of the gap between these two layers is exaggerated to provide a clearer view of how embolic members are inserted within this gap. In this example, guidewires 106 are attached to inner mesh structure 104 before the stent is inserted in the parent vessel and catheter 301 is guided to the inner mesh structure 104 by means of these guidewires.

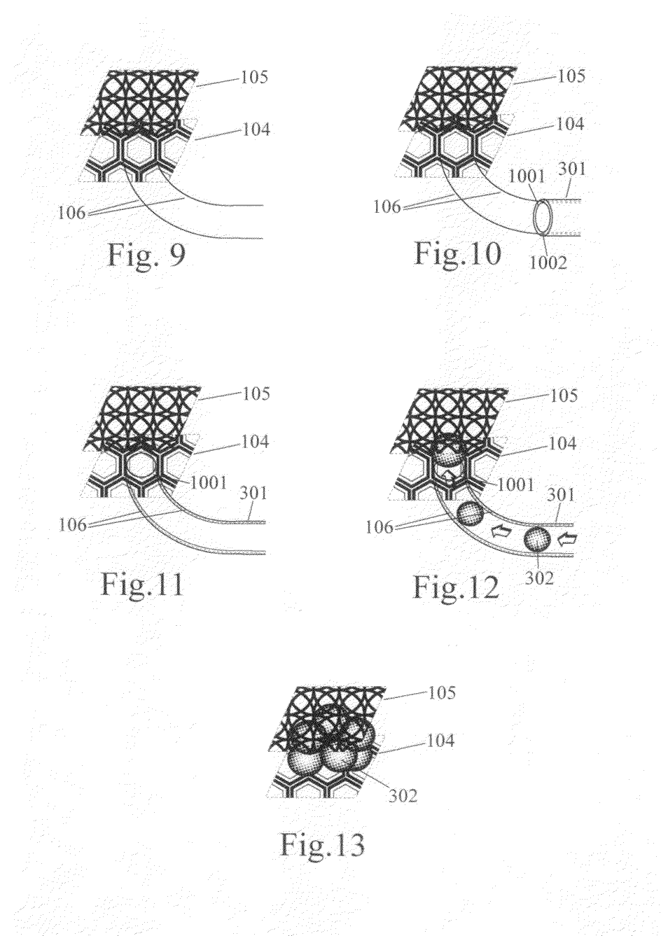

FIG. 9 shows a close-up view of guidewires 106 attached to the inside surface of a hexagonal opening in inner mesh structure 104. FIG. 9 also shows outer flexible layer 105. FIG. 9 corresponds to a close-up view of a small area of FIGS. 1 and 2, the area in which guidewires 106 are attached to inner mesh structure 104. FIG. 10 shows a close-up view of the distal end 1001 of catheter 301 as it slides along guidewires 106 toward inner mesh structure 104. The other (proximal) end of catheter 301 remains outside the person's body. There are two holes, including 1002, that run longitudinally through opposite sides of the wall of catheter 301 and contain guidewires 106, enabling catheter 301 to slide along guidewires 106. FIG. 11 shows a close-up view of the distal end 1001 of catheter 301 after it has completely slid along guidewires 106 to reach inner mesh structure 104 and be aligned with one hexagonal opening of this structure.

FIG. 12 shows a close-up view of embolic members (including 302) being propelled through catheter 301 by a flow of sterile saline solution. In this example, the embolic members are micro-sponges that expand upon ejection from the catheter into the gap between the inner mesh structure 104 and outer flexible layer 105. FIG. 12 corresponds to a close-up view of a small area of FIGS. 3 and 4, the area in which the guidewires 106 are attached to the inner mesh structure 104.

FIG. 13 shows a close-up view of a plurality of embolic members having been inserted into the gap between the inner mesh structure 104 and outer flexible layer 105. Also, guidewires 106 and catheter 301 have been detached and withdrawn. FIG. 13 corresponds to a close-up view of a small area of FIGS. 7 and 8, the area in which the guidewires were attached to the inner mesh structure.

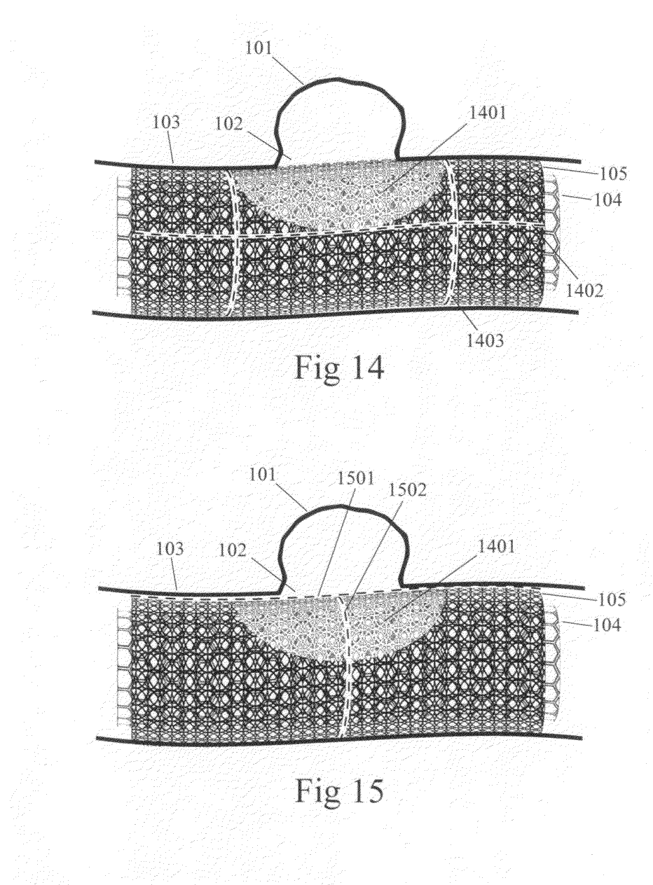

FIGS. 14 and 15 show an opaque side view of two examples of this stent that feature an outer flexible layer with differential flexibility. Having a stent with one area of the outer flexible layer that has greater flexibility and placing this area over the aneurysm neck has two advantages. First, it facilitates insertion of a substantial mass of embolic members into the gap between the inner mesh and the outer flexible layer in the area of the aneurysm neck in order to thoroughly occlude the aneurysm neck. Second, although the walls of the parent vessel resist migration of embolic members through the gap away from the aneurysm neck area, having less flexibility of the outer layer outside the aneurysm neck area provides additional resistance to possible migration of embolic members.

Specifically, FIGS. 14 and 15 show a stent, with an inner structural mesh 104 and an outer flexible net 105, having been inserted into parent vessel 103 of aneurysm 101 with aneurysm neck 102. FIGS. 14 and 15 also show a saddle-shaped area 1401 of the outer flexible net that has greater flexibility than the rest of the net. This saddle-shaped area with greater flexibility is positioned to cover the aneurysm neck when the stent is placed and expanded.

In FIGS. 14 and 15, the stent also features radioopaque lateral and longitudinal lines that help the operator to align the saddle-shaped area with the aneurysm neck during placement and expansion of the stent. In FIG. 14, the saddle-shaped area 1401 is identified for the operator by radioopaque longitudinal lines (including 1402) and lateral circumferential lines (including 1403) that intersect the outer boundaries of the saddle-shaped area. In this example, the operator positions the stent so that the aneurysm neck is centered, in each direction, between these radioopaque lines. In FIG. 15, the saddle-shaped area 1401 is identified by radioopaque longitudinal line 1501 and lateral circumferential line 1502 that intersect the center of the saddle-shaped area. In this example, the operator positions the stent so that the intersection of these lines is centered within the aneurysm neck.

In the examples shown in FIGS. 14 and 15: there is only one area of the outer flexible net with higher flexibility, this area is saddle-shaped, and this area spans approximately 15% of surface area of the stent. In other examples: there may be more than one area with higher flexibility to address multiple aneurysms, the area may have a different shape, and the area may span a higher or lower percentage of the surface area of the stent. In these examples, the radioopaque lines are lateral circumferential and longitudinal lines. In other examples, the radioopaque lines may trace the exact perimeter of the higher-flexibility area.

I claim:

1. A device that is inserted into the parent vessel of an aneurysm in order to reduce blood flow to the aneurysm, comprising:
an inner structure that can be expanded from a compressed state to a resilient expanded state within the parent vessel of the aneurysm;
an outer flexible layer that covers all or part of the inner structure; and
embolic members placed and retained in the gap between the inner structure and the outer flexible layer in the area of the aneurysm neck in order to reduce blood flow to the aneurysm.

2. The device in claim 1 wherein the inner structure is a blood-permeable mesh that is expanded by inflation of a balloon or self-expands when released from a catheter.

3. The device in claim 1 wherein the outer flexible layer is a blood-permeable net, mesh, or fabric.

4. The device in claim 1 wherein the outer flexible layer is a blood-impermeable liner.

5. The device in claim 1 wherein the embolic members are selected from the group consisting of: sponges; gels; beads; threads; and coils.

6. The device in claim 1 wherein the embolic members are positioned after insertion of the device into the parent vessel.

7. The device in claim 1 wherein the embolic members are delivered by saline flow within a catheter.

8. The device in claim 1 wherein the embolic members are retained in the gap between the inner structure and the outer layer because the embolic members expand after insertion into the gap.

9. The device in claim 1 wherein the embolic members are retained in the gap between the inner structure and the outer layer because the embolic members are inserted through one or more openings in the inner structure that are closed after the embolic members are inserted into the gap.

10. The device in claim 1 wherein: an area of the outer flexible layer has high flexibility compared to other areas of the outer flexible layer, this high-flexibility area is identified by radioopaque lines, and this high-flexibility area is positioned to cover the neck of an aneurysm.

11. A device that is inserted into the parent vessel of an aneurysm in order to reduce blood flow to the aneurysm, comprising:
an inner structure that can be expanded from a compressed state to a resilient expanded state within the parent vessel of the aneurysm, wherein this inner structure is a mesh or other blood-permeable structure;
an outer flexible layer that covers all or part of the inner structure, wherein this outer flexible layer is a net, mesh, fabric, other blood-permeable layer, blood- impermeable liner, or other blood-impermeable layer; and
embolic members placed and retained in the gap between the inner structure and the outer layer in the area of the aneurysm neck in order to reduce blood flow to the aneurysm after insertion of the device into the parent vessel, wherein these embolic members are selected from the group consisting of sponges; gels; beads; threads; and coils.

12. The device in claim 11 wherein the embolic members are delivered by saline flow within a catheter.

13. The device in claim 11 wherein the embolic members are retained in the gap between the inner structure and the outer layer because the embolic members expand after insertion into the gap.

14. The device in claim 11 wherein the embolic members are retained in the gap between the inner structure and the outer layer because the embolic members are inserted through one or more openings in the inner structure that are closed after the embolic members are inserted into the gap.

15. The device in claim 11 wherein: an area of the outer flexible layer has high flexibility compared to other areas of the outer flexible layer, this high-flexibility area is identified by radioopaque lines, and this high-flexibility area is positioned to cover the neck of an aneurysm.

16. A device that is inserted into the parent vessel of an aneurysm in order to reduce blood flow to the aneurysm, comprising:
an inner structure that can be expanded from a compressed state to a resilient expanded state within the parent vessel of the aneurysm, wherein this inner structure is a resilient mesh or other resilient blood-permeable structure;
an outer flexible layer that covers all or part of the inner structure, wherein this outer flexible layer is a net, mesh, fabric, or other blood-permeable layer; and
embolic members placed and retained in the gap between the inner structure and the outer layer in the area of the aneurysm neck in order to reduce blood flow to the aneurysm, wherein these embolic members are selected from the group consisting of: sponges; gels; beads; threads; and coils.

17. The device in claim 16 wherein the embolic members are delivered by saline flow within a catheter and inserted into the gap between the inner structure and outer layer of the device in the area of the aneurysm neck.

18. The device in claim 16 wherein the embolic members are retained in the gap between the inner structure and the outer layer because the embolic members expand after insertion into the gap.

19. The device in claim 16 wherein the embolic members are retained in the gap between the inner structure and the outer layer because the embolic members are inserted through one or more openings in the inner structure that are closed after the embolic members are inserted into the gap.

20. The device in claim 16 wherein: an area of the outer flexible layer has high flexibility compared to other areas of the outer flexible layer, this high-flexibility area is identified by radioopaque lines, and this high-flexibility area is positioned to cover the neck of an aneurysm.
